(19) United States
(12) Patent Application Publication
(10) Pub. No.: US 2026/0256569 A1
(43) Pub. Date: Sep. 3, 2026

(54) SYSTEMS AND METHODS FOR PREPARING A TRANSPLANT SITE FOR RECEIVING FOLLICULAR TRANSPLANTS AND FOR EFFECTUATING FOLLICULAR TRANSPLANTS

(71) Applicant: The General Hospital Corporation, Boston, MA (US)

(72) Inventor: William Gerald Austen, Jr., Boston, MA (US)

(21) Appl. No.: 18/878,631

(22) PCT Filed: Jun. 23, 2023
(86) PCT No.: PCT/US2023/068930

§ 371 Date: Dec. 23, 2024

Related U.S. Application Data
(60) Provisional application No. 63/366,991, filed on Jun. 24, 2022.

(51) Int. Cl. A61F 2/10; A61B 17/00; A61B 17/32; A61B 17/3205; A61B 17/34

(52) U.S. Cl. CPC A61F 2/10; A61B 17/32053; A61B 17/34; A61B 2017/00752; A61B 2017/320064

(57) ABSTRACT

An apparatus and method for removing dermal tissue from a dermis layer. The apparatus can include a hollow needle with a proximal end, a distal end, and a central lumen extending from the proximal end through the distal end. Properties of the hollow needle can be selected such that the hollow needle can be inserted into dermal tissue to remove a portion of tissue from a transplant site in preparation for the insertion of a hair follicle from a donor site. Some apparatus can include a plurality of hollow needles and/or a reciprocating arrangement to mechanically advance and withdraw the one or more hollow needles.

### CROSS REFERENCE TO RELATED APPLICATIONS

This application claims the benefit of U.S. Provisional Patent Application Ser. No. 63/366,991, filed Jun. 24, 2022, which is incorporated herein by reference for all purposes.

### BACKGROUND INFORMATION

The present disclosure relates to systems and methods for preparing a transplant site for implanting follicular units, e.g., hair follicles, and for performing a transplant.

Follicular extraction is a procedure in which a sample hair follicle and, often, surrounding tissue is removed from a donor site. The removed hair follicle can then be implanted at an implantation site in a transplant site, or graft site. To prepare the transplant site or graft site, incisions are typically made that then receive the hair follicle and any surrounding tissue. Then, the transplant/graft site is given time to recover from the procedure and embrace the transplanted follicles. However, as it is generally impractical to transplant the volume of hair follicles required to address substantial hair loss in a single procedure, a patient must undergo multiple transplant procedures. That is, once the transplant site has healed from the prior transplant procedure, the patient undergoes a subsequent procedure until the overall thickness of the hair is sufficient.

It is not uncommon that a patient with substantial hair loss will require multiple transplant procedures, each procedure or session interrupting normal life and inflicting trauma on both the donor and the transplant sites. However, it is generally accepted that the tissue of the transplants site, in particular, limits the amount of hair follicles that can be effectively transplanted in a single procedure. For example, the incisions made to receive the transplanted follicles must be separated by tissue. Also, that tissue may be bunched or pushed to accommodate the transplanted hair follicles. Thus, the time between procedures facilitates time to heal the incisions, accept the transplanted hair follicles, and accommodate the total tissue.

Thus, it would be desirable to have systems and methods that would increase the efficiency of hair transplants, for example, by increasing the number of transplanted follicles accommodated during a single procedure and/or reducing the time for the transfer site to accept the transplants and be in condition to receive a subsequent transplant procedure.

### SUMMARY OF THE DISCLOSURE

The present disclosure describes exemplary configurations of methods and devices for preparing a transplant site to receive transplanted hair follicles. Furthermore, the present disclosure provides systems and methods for affecting the implantation of hair follicles from a donor site to the transplant site in a manner that facilitates hair transplant densities not previously available. Such methods and apparatus can remove tissue from the transplant site in a manner that allows for high densities of hair follicles to be received without binding the surrounding tissue.

In accordance with one aspect of the disclosure, a method for hair transplantation is provided. The method can include: a) inserting a hollow needle into dermal tissue of a transplant site; b) capturing a portion of dermal tissue from a dermis layer within the central lumen; c) withdrawing the hollow needle along with the portion of dermal tissue, forming a void in the dermis layer; and d) repeating steps a) through c) a plurality of times to create a plurality of voids in the dermis layer. The hollow needle can have a cutting tip and a central lumen with a central lumen diameter between about 500 nm and about 0.5 mm. The method can further include the step of inserting hair follicles into the plurality of voids in the dermis layer of the transplant site without creating bunching of the dermis layer. In some aspects of the disclosure, the hollow needle can contain a protrusion within the central lumen, which can be configured to capture the portion of the dermal tissue in the capturing step. In another aspect of the disclosure, repeating steps a through c in step d can be performed in parallel with a multi-needle apparatus that can include a plurality of hollow needles.

In some aspects of the disclosure, the method for hair transplantation can include inserting the hollow needle into dermal tissue of a donor site; capturing a portion of dermal tissue with a hair follicle within the central lumen; and withdrawing the hollow needle along with the portion of dermal tissue.

In other aspects of the disclosure, the grip can be attached to the hollow needle and can define a maximum insertion distance between a lower grip surface and a most distal part of the cutting tip. The grip can be configured to limit the distance the hollow needle can extend into the dermal tissue in the inserting step. The grip can be configured to be moved relative to and along the hollow needle to modify the maximum insertion distance.

According to another embodiment of the disclosure, an apparatus for removing dermal tissue from a transplant site to receive hair transplants is provided. The apparatus can include a hollow needle with a proximal end, a distal end, and a central lumen extending from the proximal end through the distal end. The distal end can have an opening aligned with the central lumen to define a cross-section in the range of about 500 nm to about 0.5 mm. The cutting tip can be configured to cut into the dermal tissue to allow the dermal tissue to extend into the central lumen and to excise the dermal tissue from the transplant site to create a void sized to counteract bunching of the dermal tissue after receiving hair transplants. A grip can be coupled to the proximal end of the hollow needle and can be configured to control the hollow needle to cut and excise the dermal tissue.

Some aspects of the disclosure can further include a cutting tip at the distal end that is obliquely angled relative to a longitudinal axis of the central lumen. The angle of the cutting tip can be about 45 degrees.

In other aspects of the disclosure, the grip can define a maximum insertion distance between a lower grip surface and a most distal part of the cutting tip. The grip can be configured to limit the distance the hollow needle can extend into the dermal tissue. The maximum insertion distance can be the length of a portion of a hair follicle within the dermal tissue. The maximum insertion distance cam be less than 5 mm. The grip can be movable relative to and along the hollow needle to alter the maximum insertion distance.

In some aspects of the disclosure, the apparatus can further include a protrusion extending inward from the hollow needle into the central lumen. The protrusion can be configured to engage the dermal tissue entering the central lumen and retain the dermal tissue in the central lumen as the hollow needle is withdrawn from the transplant site to excise the dermal tissue from the transplant site.

In another aspect of the disclosure, the central lumen can extend along a longitudinal axis of the hollow needle and the central lumen diameter can decrease in dimension along the longitudinal axis from the cutting tip in a direction toward the proximal end.

In some aspects of the disclosure, the cross-section of the central lumen can be diamond shaped.

In other aspects of the disclosure, the apparatus can include a plurality of hollow needles, wherein the grip can form a substrate having a plurality of holes in which the plurality of hollow needles are affixed. The plurality of needles can be arranged in a rectangular pattern and spaced equidistantly in the substrate.

According to another embodiment of the disclosure, an apparatus for removing dermal tissue from a transplant site to receive hair transplants is provided. The apparatus can include a hollow needle with a proximal end, a distal end, and a central lumen extending from the proximal end through the distal end. The distal end can include a cutting tip with an opening diameter in the range of about 500 nm to about 0.5 mm and can be configured to cut into the dermal tissue to allow the dermal tissue to extend into the central lumen and to excise the dermal tissue from the transplant site. The apparatus can further include a substrate in which the hollow needle is affixed, a housing, and a linkage system coupled to the housing. Further, the substrate can be configured to translate within the housing upon activation of the linkage system, which can extend the hollow needle out of the housing.

In some aspects of the disclosure, the linkage system can include a slider-crank linkage that is activated by a switch on the housing. The switch can rotate a crank at least half a full revolution per activation.

In another aspect of the disclosure, the apparatus can include a plurality of needles affixed to the substrate.

### BRIEF DESCRIPTION OF THE DRAWINGS

Further objects, features and advantages of the present invention will become apparent from the following detailed description taken in conjunction with the accompanying figures showing illustrative examples, results and/or features of embodiments of the present invention, in which:

FIG. 1A includes an isometric view of a transplant site on a human scalp and close-up views of the transplant site during and after a transplant procedure;

FIG. 1B includes isometric views and close-up views of a transplant site on a human scalp during and after a transplant procedure with a hollow needle apparatus according to an embodiment of the invention;

FIG. 2A is an isometric view of a hollow needle apparatus according to another embodiment of the invention;

FIG. 2B is a side elevation view of the hollow needle apparatus of FIG. 2A;

FIG. 3A is a close-up view of the hollow needle apparatus of FIG. 2A;

FIG. 3B is a cross-sectional view of the hollow needle apparatus of FIG. 2A along line 3B-3B of FIG. 3A;

FIG. 4A is a close-up view of a hollow needle apparatus according to another embodiment of the invention;

FIG. 4B is a cross-sectional view of the hollow needle apparatus of FIG. 4A along line 4B-4B of FIG. 4A;

FIG. 5A is a close-up view of a hollow needle apparatus according to another embodiment of the invention;

FIG. 5B is a cross-sectional view of the hollow needle apparatus of FIG. 5A along line 5B-5B of FIG. 5A;

FIG. 6 is a cross-sectional view of the hollow needle of FIG. 2A adjacent dermal tissue of a transplant site according to an embodiment of the invention;

FIG. 7 is a cross-sectional view of the hollow needle of FIG. 2B inserted in a transplant site of dermal tissue according to an embodiment of the invention;

FIG. 8 is a cross-sectional view of the hollow needle apparatus of FIG. 2A withdrawing from the dermal tissue of the transplant site according to an embodiment of the invention;

FIG. 9 is a cross-sectional view of the hollow needle apparatus of FIG. 2A inserted in donor site;

FIG. 10 is a cross-sectional view of the hollow needle apparatus of FIG. 2A with a hair follicle aligned above the transplant site;

FIG. 11 is a cross-sectional view of the hollow needle apparatus of FIG. 2A withdrawing from the transplant site after implanting the hair follicle;

FIG. 12 is a cross-sectional view of a hollow needle apparatus according to another embodiment of the invention;

FIG. 13 is a cross-sectional view of the hollow needle apparatus of FIG. 12 inserted in a transplant site of dermal tissue;

FIG. 14 is an top isometric view of a hollow needle apparatus according to another embodiment of the invention;

FIG. 15 is a side elevation view of the hollow needle apparatus of FIG. 14;

FIG. 16 is a cross-sectional view of a hollow needle apparatus according to another embodiment of the invention;

FIG. 17 is a cross-sectional view of a hollow needle apparatus according to another embodiment of the invention;

FIG. 18 is a block diagram illustrating a method for follicular transplantation according to an embodiment of the invention.

Throughout the drawings, the same reference numerals and characters, unless otherwise stated, are used to denote like features, elements, components, or portions of the illustrated embodiments. Moreover, while the present disclosure will not be described in detail with reference to the figures, it is done so in connection with the illustrative embodiments and is not limited by the particular embodiments illustrated in the figures and the appended claims.

### DETAILED DESCRIPTION

Before any embodiments of the invention are explained in detail, it is to be understood that the invention is not limited in its application to the details of construction and the arrangement of components set forth in the following description or illustrated in the following drawings. The invention is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless specified or limited otherwise, the terms “mounted,” “connected,” “supported,” and “coupled” and variations thereof are used broadly and encompass both direct and indirect mountings, connections, supports, and couplings. Further, “connected” and “coupled” are not restricted to physical or mechanical connections or couplings.

As used herein, unless otherwise defined or limited, directional terms are used for convenience of reference for discussion of particular figures or examples. For example, references to upper, lower, upward, downward, or other directions may be used to discuss aspects of a particular example or figure, but do not necessarily require similar orientation or geometry in all installations or configurations.

The following discussion is presented to enable a person skilled in the art to make and use embodiments of the invention. Various modifications to the illustrated embodiments will be readily apparent to those skilled in the art, and the generic principles herein can be applied to other embodiments and applications without departing from embodiments of the invention. Thus, embodiments of the invention are not intended to be limited to examples shown, but are to be accorded the widest scope consistent with the principles and features disclosed herein. The following detailed description is to be read with reference to the figures, in which like elements in different figures have like reference numerals. The figures, which are not necessarily to scale, depict selected embodiments and are not intended to limit the scope of embodiments of the invention. Skilled artisans will recognize the examples provided herein have many useful alternatives and fall within the scope of embodiments of the invention.

Some of the discussion below describes an apparatus for removing a portion of dermal tissue. The context and particulars of this discussion are presented as examples only. For example, embodiments of the disclosed invention can be configured in various ways, including with other shapes and arrangements of elements. The context and particulars of this discussion are presented as examples only.

Hair transplantation procedures can generally involve removing small grafts (e.g., “punch” grafts) from a donor site that includes hair follicles. Further, slits are cut into the bald or thinning area of the scalp (i.e., the transplant site) with a small knife or similar device and the small grafts are inserted into the slits. Challenges when performing the transplant of hair grafts can include both overcrowding versus sparce coverage over a transplant site. For example, overcrowding can occur when performing multiple follicular implants in transplant site causes the surrounding, existing, tissue to bunch up, creating an uneven surface along the implant site. Alternatively, too much space between the follicular implants (i.e., sparce coverage) can leave gaps between implants in the transplant site and an incomplete, thin, appearance, thereby necessitating subsequent transplant procedures.

The systems and methods provided herein enable transplantation procedures that reduce or avoid creating negative cosmetic effects in the transplant site, such as bunching or sparce implant coverage, while reducing the need for multiple, or serial transplant procedures. Thus, the systems and methods provided herein facilitate a more efficient, safe, and consistent follicular transplant of one or more hair follicles. For example, in some configurations, a hollow needle is configured to remove a portion of dermal tissue from the implant or transplant site prior to the implantation of the graft. In some configurations, an apparatus is provided that includes multiple hollow needles that are configured to remove dermal tissue in a predetermined pattern within a transplant site. In other configurations, a system is provided that includes a hollow needle that can remove a portion of dermal tissue from a transplant site, remove a graft from a donor site, and implant the graft into the transplant site in the location of the removed dermal tissue portion.

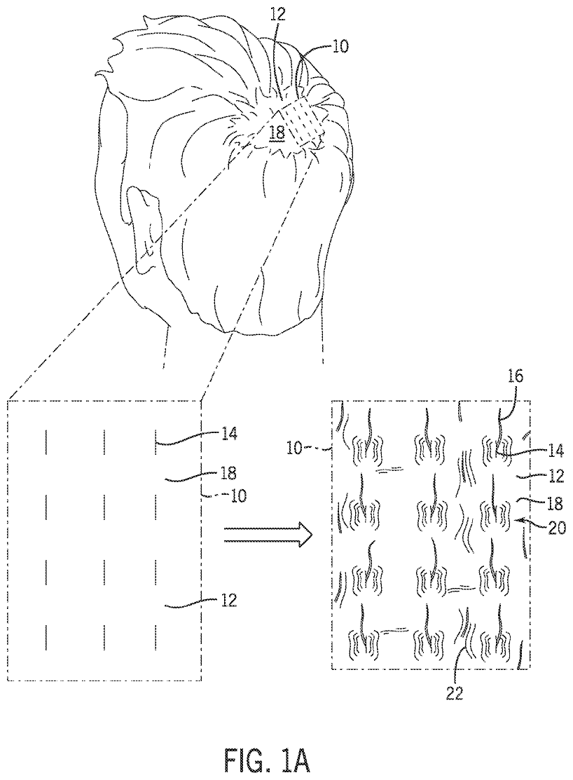

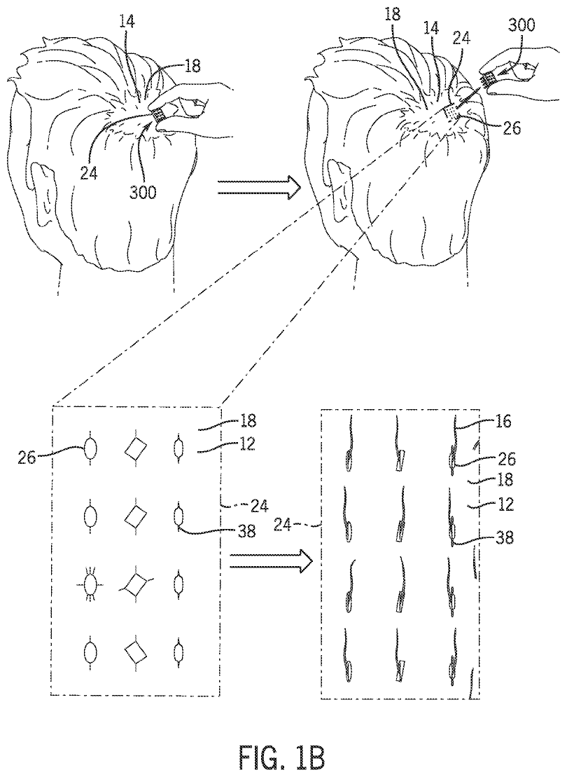

FIG. 1A shows a transplant site 10 on a human scalp 12 during and after a hair transplant procedure. In conventional hair transplant procedures, a scalpel, or other incision tool, is used to cut slits 14 within the human scalp 12, which are then opened to create a space in which a hair follicle 16 can be placed. However, as shown in FIG. 1A, the slits 14 are not able to completely close, because the hair follicles 16, and any dermal tissue surrounding the hair follicle 16 taken from a donation site, adds material that was not previously present at the transplant site 10. A consequence of this is that the implanted hair follicles 16 urge the slits 14 open and further urge the dermal tissue 18 surrounding the slits 14 away from the respective slit 14 against adjacent dermal tissue with no room for the additional dermal tissue to go. The end result is areas of bunched dermal tissue 20 throughout the transplant site 10 surrounding each of the implanted hair follicles 16. When observed over an entire transplant site (e.g., the transplant site 10), areas of bunched dermal tissue 20 can create ripples 22 along the scalp 12. The ripples 22 create an unnatural looking surface on the scalp 12 and are a detriment to the intended outcome of providing a natural appearing hair transplant site 10. Therefore, a minimum spacing between the implanted hair follicles 16 must be maintained to avoid areas of bunched dermal tissue 20 and the resulting ripples 22. Further, as a result of such spacing, fewer hair follicles 16 can be implanted in a transplant site 10 during any one transplant operation. Therefore, more follicular transplant procedures are required to ultimately achieve the desired density of hair follicles 16 within the transplant site, which means more overall operating time, more overall cost, more overall recovery time, and an overall longer treatment schedule.

FIG. 1B shows a multi-needle apparatus 300 that can be inserted into and then removed from dermal tissue 18 in a transplant site 24 on a scalp 12 of a human patient and also illustrates close-up views of the transplant site 24, according to one aspect of the disclosure. As will be described in more detail herein, the multi-needle apparatus 300 is one example configuration for a needle apparatus configured to perform a follicular transplantation process also described herein. Other configurations of needle apparatus will be described herein, but the disclosure should not be construed as limited to only these configurations.

Continuing, as shown in the close-up view in the lower left of FIG. 1B, a hollow needle or a plurality of hollow needles on a multi-needle apparatus, as discussed in more detail below, can be used to create a plurality of core voids 26 in the dermal tissue 18 in the treatment site 24. That is, a dermal tissue removal procedure can be performed as discussed further below. In some embodiments, each core void in the plurality of core voids 26 can have a similar cross-sectional shape. In some embodiments, the cross-section shapes of core voids in the plurality of core voids 26 can be different. FIG. 1B provides a few non-limiting examples of possible cross-sectional shapes of the core voids 26. As will be described, shape or shapes of the core voids 26 are dictated by a cross-sectional shape of the hollow needle(s) used for the dermal tissue removal procedure. Most commonly, only one shape of core voids 26 will be created in a given patient or at least a given treatment site 24. Here, however, a variety of shapes are shown simply for illustrative purposes. Furthermore, though a few different shapes are illustrated, these shapes should not be considered exhaustive or limiting. It is contemplated that the cross-sectional shapes can include other shapes not shown.

As shown in the bottom right of FIG. 1B, the core voids 26 allow the hair follicles 16 to be arranged in a condensed transplantation site compared to the site shown in FIG. 1A, while limiting or avoiding the bunched dermal tissue 20 of FIG. 1A. Thus, as illustrated in FIG. 1B, the systems and methods provided herein allow for a clinician to achieve a greater follicular transplant density within the transplant site without tissue binding or bunching, which reduces overall operating time, reduces overall cost, reduces overall recovery time, and reduces the overall treatment schedule.

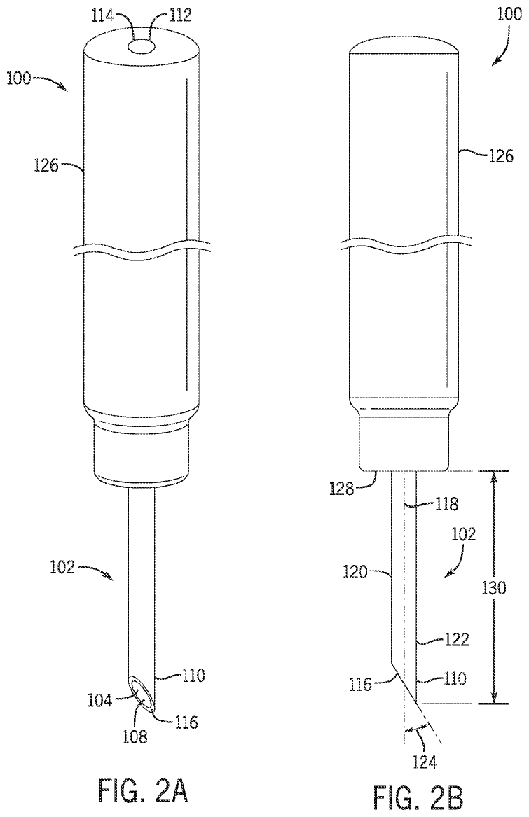

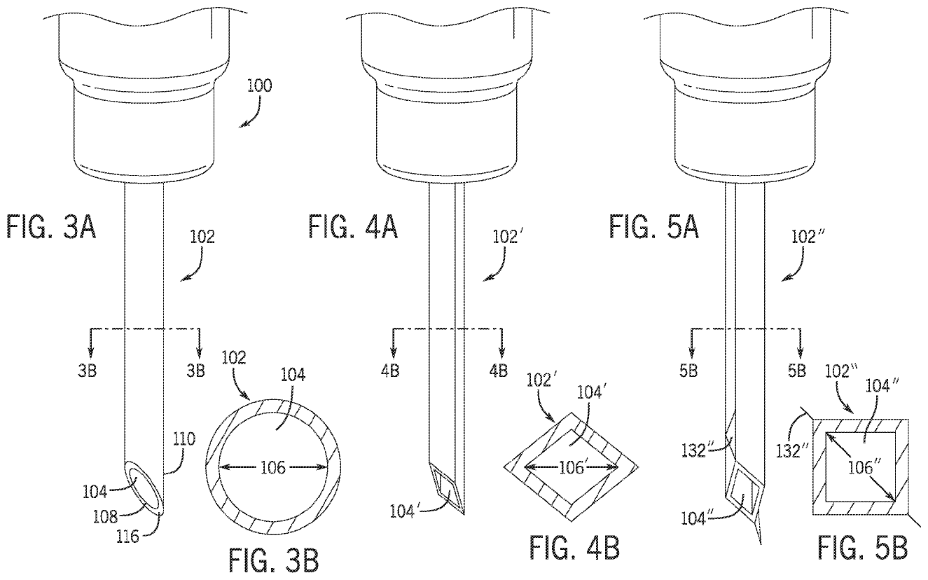

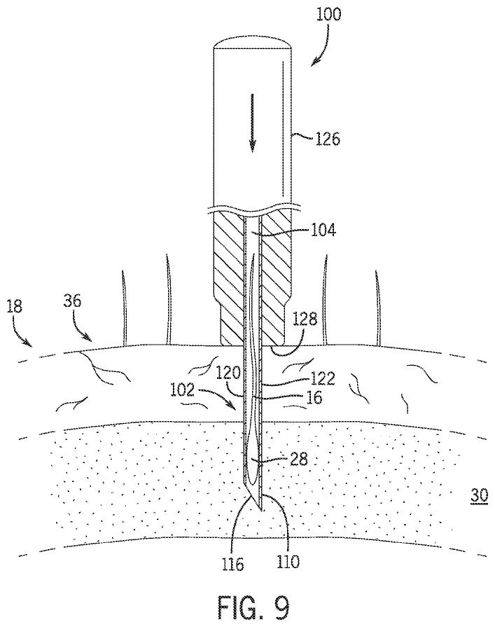

FIGS. 2A, 2B, 3A, and 3B illustrate a hollow needle apparatus 100 according to one aspect of the disclosure. The hollow needle apparatus 100 is configured to remove a portion of tissue (e.g., the human dermal tissue 18 shown in FIG. 1B). The hollow needle apparatus 100 can also be configured to remove a hair follicle 16 (as shown in FIG. 9). The hollow needle apparatus 100 can have a hollow needle 102 with a central lumen 104 with a central lumen diameter 106 (shown in FIG. 3B), a distal opening 108 at a distal end 110 of the hollow needle 102, and a proximal opening 112 at a proximal end 114 of the hollow needle 102. The distal opening 108 is aligned with the central lumen 104 and defines a cross-section of the central lumen 104. The distal end 110 can be configured with a cutting tip 116 that can be sharpened and/or angled to facilitate insertion of the needle 100 into the dermal tissue 18. In some configurations, the cutting tip 116 can have a wedge shape formed from, for example, grinding the distal end 110 of the hollow needle 102. As shown in FIG. 2B, for example, the cutting tip 116 is obliquely angled relative to the longitudinal axis 118 at an angle 124. The angle 124 of the edge 116 in relation to the longitudinal axis 118 can be between about 30 degrees and 60 degrees, for example, which can provide tissue-selective penetration characteristics as described in this disclosure. In some configurations, the angle 124 can be about 45 degrees. The cutting tip 116 can define a short side 120 and a long side 122 of the hollow needle 102. In some configurations, the cutting tip can be formed as a 2-prong or a multi-prong cutting tip.

As shown in the FIG. 3B, the hollow needle 102 and the central lumen 104 have substantially circular cross-sections. However, other cross-sectional shapes of hollow needles and central lumens are contemplated. For example, other cross-sectional shapes can include oval, diamond (hollow needle 102′, central lumen 104′, and central lumen diameter 106′ shown in FIGS. 4A and 4B), square (hollow needle 102″, central lumen 104″, and central lumen diameter 106″ shown in FIGS. 5A and 5B), triangular, or lenticular shapes. In configurations other than circular cross-section hollow needles, the central lumen diameter dimension can be defined as the length of a line extending between two furthest opposed points on the shape that also passes through the geometric center of the shape. Additionally, or alternatively, in some configurations, the central lumen diameter at or near the distal end can different than the central lumen diameter at or near the proximal end. For example, the central lumen diameter at the distal end (e.g., at the cutting tip) can be larger than the central lumen diameter at the proximal end. In some configurations, the change in central lumen diameter along the central lumen can be linear (i.e., the change in central lumen diameter is constant along the length of the central lumen). The cross-section and central lumen diameter 106 are selected and configured to remove a portion of the dermal tissue, but leave very little, if any, visible damage thereto. In some configurations, the central lumen diameter 106 can be in the range of about 500 nm to about 0.5 mm.

Further, as shown in FIGS. 5A and 5B additional cutting features can be included on the hollow needle 102″. For example, blades 132″ can extend radially from the hollow needle 102″ at the distal end 110″. The blades 132″ can ease the insertion of the hollow needle 102″ into the dermal tissue 18 during the transplant procedure because the blades 132″ provide leading and trailing cutting edges that can relieve the stress experienced by the dermal tissue 18 when the hollow needle 102″ is inserted. Additionally, reliefs 38 (shown in FIG. 1B) formed within the dermal tissue 18 from the blade 132″ can aid implantation of the hair follicle 16 because it allows the core void 26 to be opened a little further. For example, some hair grafts containing the hair follicle 16 can have a diameter or width dimension in the range of about 0.8 mm to about 1.0 mm, which is greater than the core void 26 created by a central lumen 104 with a central lumen diameter 106 of 0.5 mm or less. The reliefs 38 can therefore allow the core void 26 to be opened wider to accept the hair graft and then close around the hair graft. The potential scaring is thereby minimized because the amount of dermal tissue 18 removed from the transplant site 24 is minimized. It is contemplated that the number, size, and location of the blades 132″ on the hollow needle 102″ can vary, for example, depending on the size of the hair graft being transplanted.

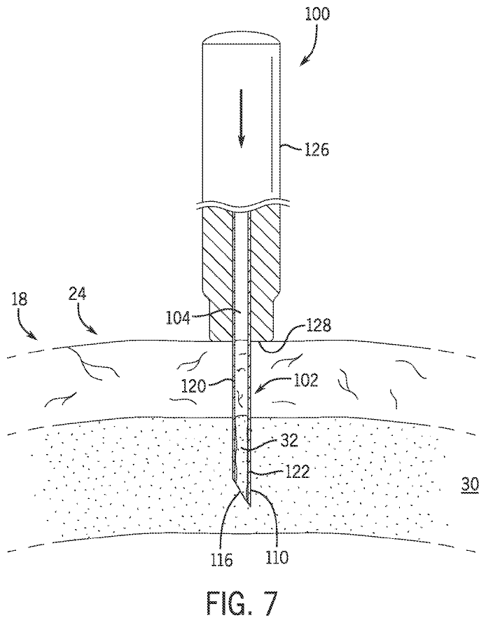

Continuing, the hollow needle apparatus 100 can be configured to be inserted into the dermal tissue 18 such that the distal end 110 of the hollow needle 102 penetrates to a depth below the approximate location of a bulb 28 of a hair follicle 16 (show in FIG. 9), typically located within the dermis layer 30, and through the dermis layer 30 into, for example, the subcutaneous fatty layer. In some configurations, as shown in FIGS. 2A and 2B, the hollow needle apparatus 100 can have a grip 126 at the proximal end 114 of the hollow needle 102. The grip 126 can facilitate holding and manipulating the hollow needle 102. The grip 126 can also act as a depth stop, limiting the depth that the hollow needle 102 can be inserted into the dermal tissue 10 (as shown in FIG. 7). The grip 126 has a lower grip surface 128, and a maximum insertion distance 130, which is defined as the distance from the lower grip surface 128 of the grip 126 to the end of the cutting tip 116 of the hollow needle 102. In configurations with a cutting tip 116 is obliquely angled relative to the longitudinal axis 118 of the hollow needle 102, the maximum insertion distance 130 is defined as the distance from the lower grip surface 128 to the cutting tip 116 along the long side 122 of the hollow needle 102. The maximum insertion distance 130 can be based on a predetermined desired maximum depth of insertion into the dermal tissue 18 (as shown in FIG. 7). In some configurations, the maximum insertion distance 130 can be less than 5 mm. In some configurations, the grip 126 can be configured to be moved relative to and along the hollow needle 102 to modify the maximum insertion distance 130.

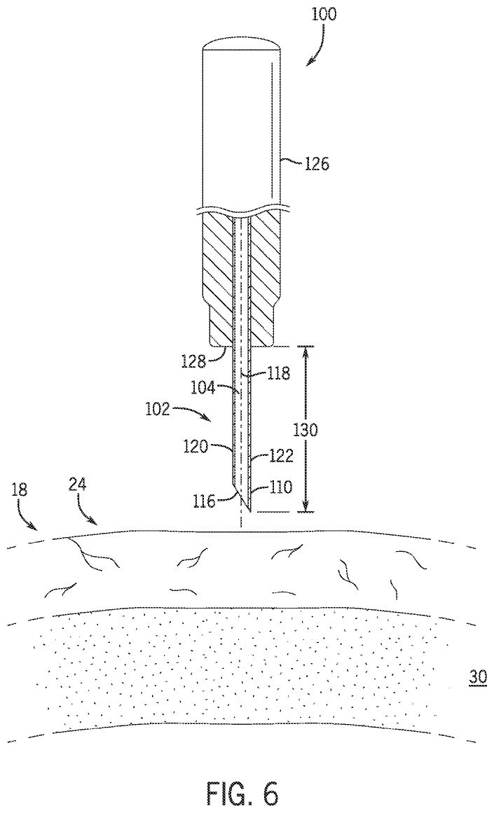

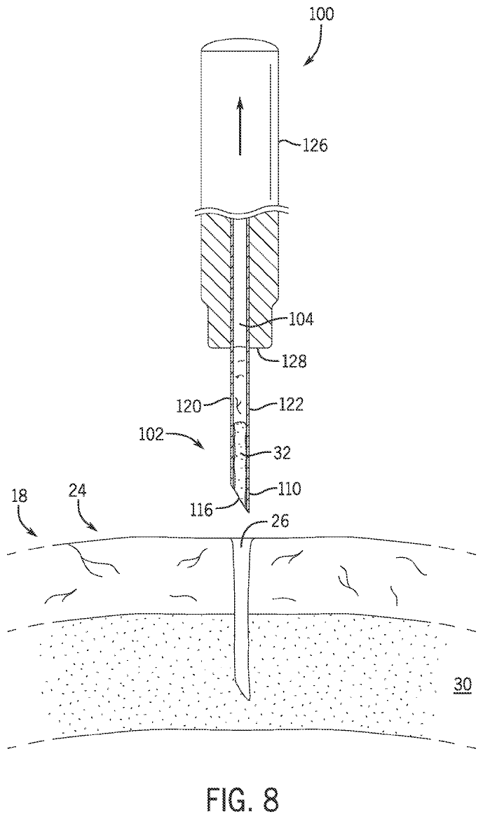

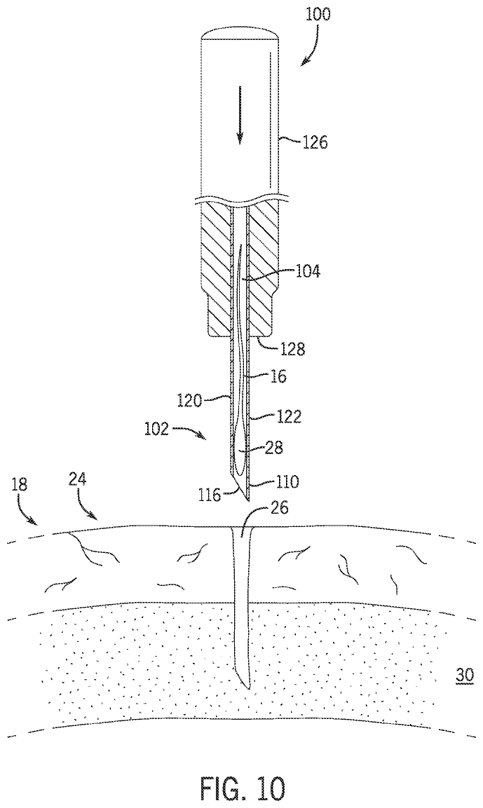

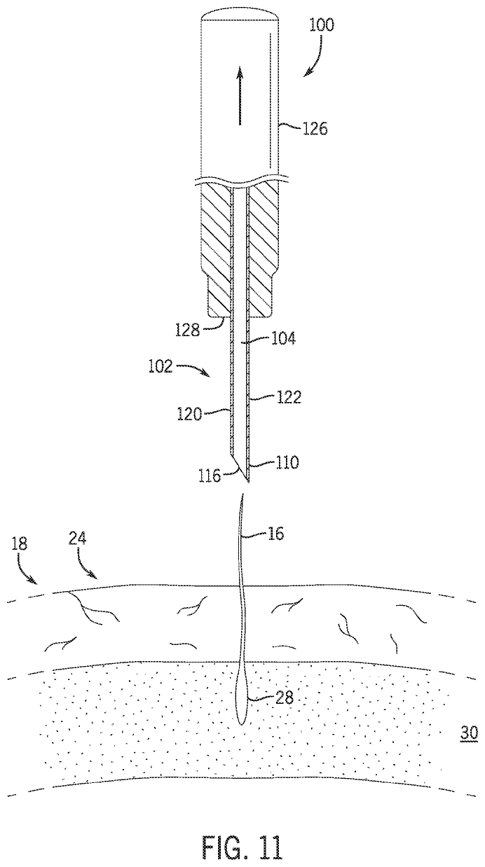

Insertion of the hollow needle 102 into the dermal tissue 18 of the transplant site 24, and subsequent removal therefrom, (shown in FIGS. 6 through 8) can remove a dermal tissue core 32 and form a microscopic hole, a core void 26 (shown in FIGS. 1B and 8), into which a transplant hair follicle 16 from a donor site 36 (shown in FIG. 9) can be implanted (shown in FIGS. 10 and 11). Removal of the dermal tissue core 32 from the transplant site 24 provides a space in which to insert a graft, thus reducing the potential for pushing, or bunching, of the dermal tissue 18 in the transplant site 24 and leaving a smooth surface along the dermal tissue 18. Further, as shown in FIG. 1B, the spacing between implanted hair follicles 16 can be reduced due to the reduction in tissue bunching. As a result of such spacing, more hair follicles 16 can be implanted in the transplant site 24 during any one transplant operation. Therefore, fewer follicular transplant procedures are required to ultimately achieve the desired density of hair follicles 16 within the transplant site 24, which means less overall operating time, less overall cost, less overall recovery time, and an overall shorter treatment schedule.

In some configurations, it is contemplated that the hollow needle apparatus 100 can be configured to remove and dispose of the dermal tissue core 32 from the transplant site 24, form the core void 26; remove the hair follicle 16 from the donor site 36; and transplant the hair follicle 16 into the core void 26 in the transplant site 24. In some configurations, one hollow needle apparatus 100 can be used for the removal and disposal of the dermal tissue core and another hollow needle apparatus 100 can be used for the removal and implantation of the hair follicle 16.

In some configurations, it is contemplated that the hollow needle apparatus 100 can further include a vacuum source (not shown) coupled to the hollow needle 102 to aid in removal of the dermal tissue core 32 from the transplant site 24 and into the central lumen 104 for disposal. It is further contemplated that the dermal tissue core 32 could be ejected from the central lumen 104 by reversing the polarity of the vacuum source and forcing air there through and out of the distal end 110. Additionally, or alternatively, the vacuum source could aid in removal and transplantation of the hair follicle 16 out of the donor site 36 and into the transplant site 24, respectively, in a similar manner.

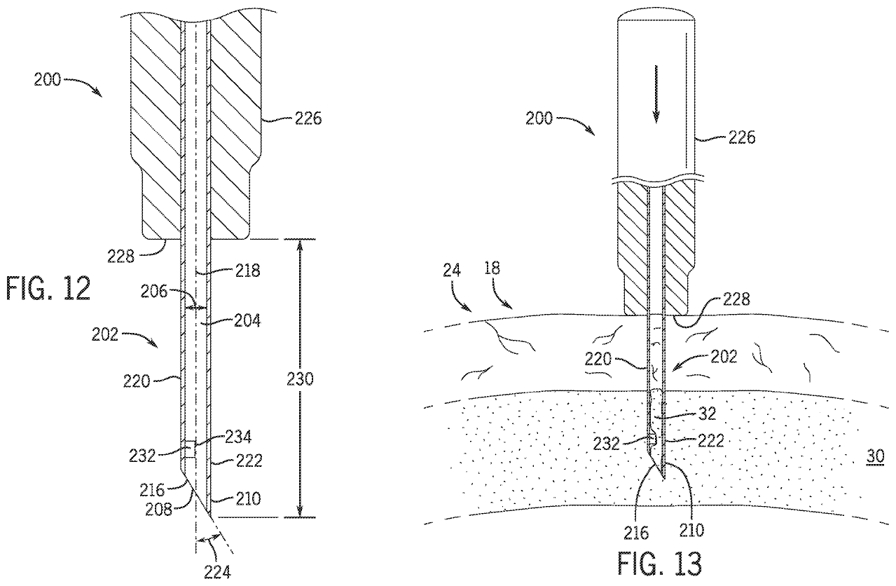

FIGS. 12 and 13 illustrate another embodiment of a hollow needle apparatus 200 according to the invention, as also can be used to remove and/or implant dermal tissue. In many aspects, the hollow needle apparatus 200 is similar to the hollow needle apparatus 100 described above and similar numbering in the 200 series is used for the hollow needle apparatus 200. For example, the hollow needle apparatus 200 has a hollow needle 202 with a central lumen 204 with a central lumen diameter 206, a distal opening 208 at a distal end 210 with a cutting tip 216 having an angle 224 relative to a longitudinal axis 218, a proximal opening (hidden) at a proximal end (hidden), a short side 220, and a long side 222, and a grip 226 with a lower grip surface 228. Further, the hollow needle apparatus 200 is configured to be insertable into dermal tissue a maximum insertion distance 230 defined between the lower grip surface 228 and the cutting tip 216 at the long side 222 of the hollow needle.

In some aspects, however, the hollow needle apparatus 100, 200 differ from each other. For example, the hollow needle apparatus 200 further includes a protrusion 232 extending inward from the hollow needle 202 into the central lumen 204 on the short side 220 and proximal to the distal end 210. The protrusion 232 may be useful to grip the dermal tissue within the central lumen 204 to ensure proper removal thereof. The protrusion 232 can be configured to at least partially occlude the central lumen 204. For example, the protrusion 232 can block less than about 50% of the cross-sectional area of the central lumen 204. In other configurations, the protrusion 232 can block less than about 30% of the cross-sectional area. In some configurations, the protrusion 232 can block more than about 10% of the cross-sectional area, or more than about 20% of the cross-sectional area. The protrusion 232 has a protrusion edge 234 provided at the distal end thereof. The protrusion edge 234 can have a substantially straight profile as illustrated in FIG. 12. Alternatively, in some configurations, the protrusion edge 234 can be curved inward or outward, toward or away from, the center of the central lumen 204. Further, in some configurations, the protrusion 232 can be one of a plurality of protrusions in a set of protrusions positioned at various locations along the central lumen 204 (e.g., spaced linearly along the central lumen 204 and/or radially spaced thereabout). In some configurations, the set of protrusions can include protrusions having different dimensions (e.g., more or less occlusive) and/or edge profiles (e.g., straight or curved).

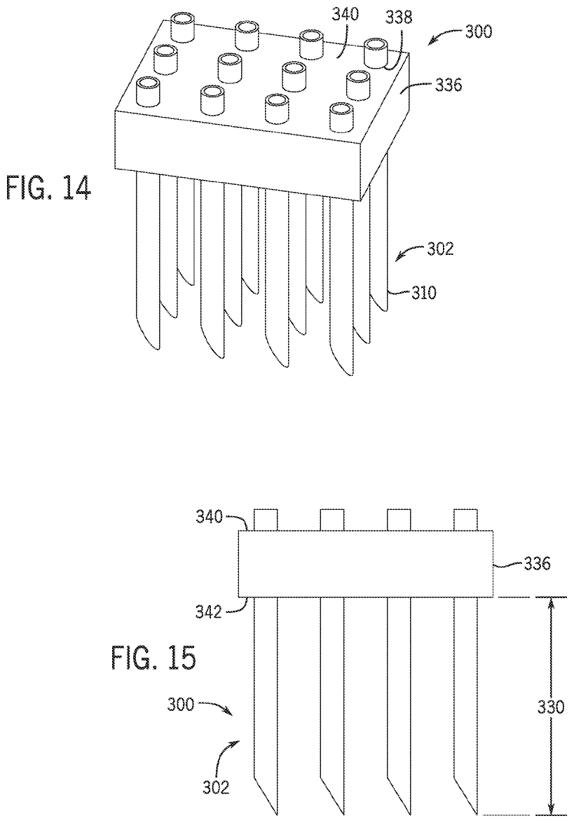

FIGS. 14 and 15 illustrate another embodiment of the invention providing the multi-needle apparatus 300 as also shown in FIG. 1B. The multi-needle apparatus 300 includes a substrate 336 with a plurality of holes 338 extending from an upper substrate surface 340 through a lower substrate surface 342 and a plurality of the hollow needles 302, each extending through a hole 338 and affixed to substrate 336 with distal ends 310 of the hollow needles 300 proximate to the lower substrate surface 342. The hollow needles 302 can be similar in design and functionality as the hollow needles 100, 200 described above. In some configurations, the hollow needles 302 can be affixed to the substrate 336 with an adhesive. As shown in FIG. 15, the lower substrate surface 342 of the substrate 336 is substantially flat and planar. However, it is contemplated that the lower substrate surface 342 can be curved or otherwise contoured to more closely follow the surface profile of the dermal tissue being treated.

The plurality of hollow needles 302 are shown extending a maximum insertion distance 330 from the substrate 336. The maximum insertion distance 330 is defined as the distance from the lower substrate surface 342 of the substrate 336 to the distal end 310 at the long side 322 of a hollow needle 302. The maximum insertion distance 330 can be based on a predetermined desired maximum depth of insertion into the dermal tissue 18 (e.g., as shown in FIG. 7 with the hollow needle 100). For example, in operations such as hair follicle transplantation, it may be determined that the needles 302 will extend into the subcutaneous fatty layer, below the dermis layer and the bulb of a hair follicle but short of entering into the muscle layer. In some configurations, the maximum insertion distance 330 can be less than 5 mm. In other configurations, the maximum insertion distance 330 can be made adjustable. For example, a plate (not shown) can be affixed to the lower substrate surface 342 of the substrate 336, effectively becoming a modified lower substrate surface and shortening the maximum insertion distance 330. It is further contemplated that based on the contour of the lower substrate surface 342 and/or the dermal tissue being treated, the maximum insertion distances 330 of each of the hollow needles 302 can be different. For example, some hollow needles 302 can extend outward farther from the lower substrate surface 342 than other hollow needles 302, thus having a longer maximum insertion distance 330.

FIG. 14 also illustrates that the plurality of hollow needles 302 can be arranged in a rectangular pattern and spaced equidistantly. In some configurations, however, the plurality of hollow needles 302 can be arranged differently. For example, the spacing between the hollow needles 302 can be varied, in a spatially random-appearing distribution. Further, it is contemplated that the number of hollow needles 302, the arrangement thereof, and the spacing therebetween can be determined based on a particular use case (e.g., the type of tissue being treated, the location of the tissue being treated on the body, the amount of tissue being treated, etc.).

The multi-needle apparatus 300 can be used just as described above with respect to the isolated hollow needles 100, 200. For example, the multi-needle apparatus 300 can be pressed into, and subsequently withdrawn from, the dermal tissue 18 to remove a dermal tissue core 32 or a hair follicle 16 (as shown in FIGS. 1A and 1B, respectively). The multi-needle apparatus 300 can also be used to implant hair follicles 16 into core voids 26 in the transplant site 24 (shown in FIG. 1B). The multi-needle apparatus 300 can facilitate removal and/or implantation of dermal tissue 18 and/or hair follicles 16 across a larger area in a single insertion/withdrawal cycle. The predetermined pattern and spacing of the plurality of hollow needles 302 can increase the efficiency of the hair transplants, for example, by increasing the number of transplanted follicles accommodated during a single procedure and/or reducing the time for the transfer site to accept the transplants and be in condition to receive a subsequent transplant procedure.

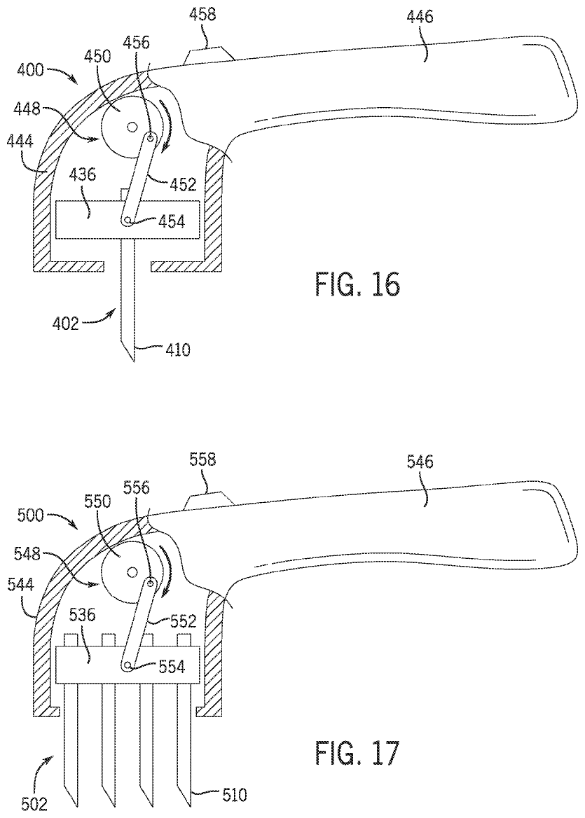

FIG. 16 illustrates another embodiment of the invention providing a reciprocating needle apparatus 400. The reciprocating needle apparatus 400 can include a hollow needle 402 extending from a substrate 436. The substrate 436 and the hollow needle 402 can be similar in design and functionality to the substrate 336 and plurality of hollow needles 302 of the multi-needle apparatus 300 described above. Further, the reciprocating needle apparatus 400 can include a housing 444 with a handle 446 and can be configured to displace the hollow needle 402 up and down along a direction parallel to the longitudinal axis of the hollow needle 402. For example, the substrate 436 could be part of a linkage system (e.g., a slider-crank linkage system 448 as shown) that is coupled to the housing 444. The system includes a crank 450 configured to rotate about a central axis and an arm 452 connected on one end to a first joint 454 on the substrate 436 and on the other end to a second joint 456 on the crank 450 spaced from the central axis so that as the crank 450 rotates. the linkage 452 pulls or pushes the substrate 436, and the hollow needle 402, up and down relative to the housing 444. In some configurations, a mechanical switch (for example, switch 458) can be activated to rotate the crank 450 at least half a full revolution per switch activation. In some configurations, the crank 450 can be rotated by a motor, or the like, and can be controlled by a switch (for example, switch 458). In some configurations, the switch 458 can be a variable speed switch (e.g., a slide potentiometer) and the motor can be a variable speed motor, whereby the speed of the motor is dependent upon the amount the switch 458 is moved between the fully open and fully closed positions. The reciprocating needle apparatus 400 can be traversed over a region of dermal tissue to be treated such that the hollow needle 402 can be repeatedly inserted and withdrawn from the dermal tissue. Automation of the insertion and withdrawal steps increases the consistency and efficacy of the procedure.

In other configurations, the reciprocating needle apparatus 400 can include a translational mechanism configured to translate the hollow needles 402 over the dermal tissue surface in one or two orthogonal directions. For example, the reciprocating needle apparatus 400 can be configured to translate the hollow needle 402 over an area of dermal tissue while the reciprocating needle apparatus 400 is held stationary with respect to the surface of the dermal tissue at the donor site 36 or the transplant site 24. In another configuration of the present disclosure, the reciprocating needle apparatus 400 can be configured to translate the hollow needle 402 along a single direction to remove or harvest tissue along one or more rows. The reciprocating needle apparatus 400 can optionally be translated over the dermal tissue surface after such rows are formed, e.g., in a direction that is not parallel to the row, to remove or harvest tissue from a larger area of the donor site 36 or the transplant site 24.

In some configurations, the housing 444 can be configured to stretch skin or other tissue when the reciprocating needle apparatus 400 is placed on the dermal tissue to be treated.

Such stretching can facilitate mechanical stabilization of the tissue, e.g., to reduce or avoid deformation of the dermal tissue while the hollow needle 402 is inserted into and withdrawn from the dermal tissue. Such stretching of the tissue can also reduce the effective size of the disrupted region of the upper tissue layers formed by the reciprocating needle apparatus 400 when the dermal tissue is allowed to relax after transplant. Alternatively, the surface of the dermal tissue to be treated can be stretched or stabilized using other techniques prior to and/or during transplant to the region in accordance with any of the exemplary embodiments described herein.

For example, a vacuum or suction source, e.g. a pump or a reservoir containing a fluid under low-pressure, can be provided in communication with a lumen of the hollow needle 402, e.g., via a conduit in communication with the proximal end of the hollow needle 402, in any of the exemplary embodiments described herein. Such low pressure, e.g., pressure less than atmospheric or ambient pressure, provided in the lumen can facilitate the removal of the portions dermal tissue when the distal end 410 of the hollow needle 402 is located within the dermis layer 30 (for example, as shown in FIG. 7 with the hollow needle apparatus 100). For example, the exemplary devices described herein can be configured to provide such a vacuum when the distal end 410 of the hollow needle 402 is at least partially inserted into the dermis layer 30, and such vacuum may be applied as the hollow needle 402 is withdrawn from the dermis layer 30. The strength of the vacuum can be selected to facilitate removal of the portions of the dermal tissue within the distal end 410 of the hollow needles 402, without causing significant damage to the dermal tissue surrounding the inserted hollow needle 402.

FIG. 17 illustrates another embodiment of a reciprocating needle apparatus 500 according to the invention, as also can be used to remove and/or implant dermal tissue. In many respects the reciprocating needle apparatus 500 is similar to the reciprocating needle apparatus 400 described above and similar numbering in the 500 series is used for the reciprocating needle apparatus 500. For example, the reciprocating needle apparatus 500 has a substrate 536, a housing 544, a handle 546, and a slider-crank linkage system 548 including a crank 550 and an arm 552 attached to the substrate 536 at a first joint 554 and the crank 550 at a second joint 556. Further, the substrate 536 is configured to be movable up and down within the housing 544 through operation of the slider-crank linkage system 548. The crank 550 can be rotated by a motor, or the like, and can be controlled by a switch (for example, switch 558). 510

In some aspects, however, the reciprocating needle apparatus 400, 500 differ from each other. For example, the reciprocating needle apparatus 500 includes a plurality of hollow needles 502, with distal ends 510, affixed to the substrate 536. As discussed above with respect to the multi-needle apparatus 300, the plurality of hollow needles 502 can facilitate removal and/or implantation of dermal tissue 18 and/or hair follicles 16 across a larger area in a single insertion/withdrawal cycle.

Any of the apparatus described herein can be configured to remove or harvest dermal tissue from a plurality of locations in any of a variety of spatial distributions, where each location can correspond to a single insertion and withdrawal of a hollow needle. For example, the dermal tissue can be removed or harvested from a plurality of locations configured as one or more rows, a regular two-dimensional pattern, a random distribution, or the like. Such exemplary patterns or spatial distributions of tissue harvesting or removal sites can be generated based on, e.g., the configuration of such one or more needles provided, the properties of the reciprocating needle apparatus, and/or the rate of translation of the reciprocating needle apparatus over the dermal tissue surface.

In some implementations, devices or systems disclosed herein can be utilized or installed using methods embodying aspects of the invention. Correspondingly, description herein of particular features or capabilities of a device or system is generally intended to inherently include disclosure of a method of using such features for intended purposes and of implementing such capabilities. Similarly, express discussion of any method of using a particular device or system, unless otherwise indicated or limited, is intended to inherently include disclosure, as embodiments of the invention, of the utilized features and implemented capabilities of such device or system.

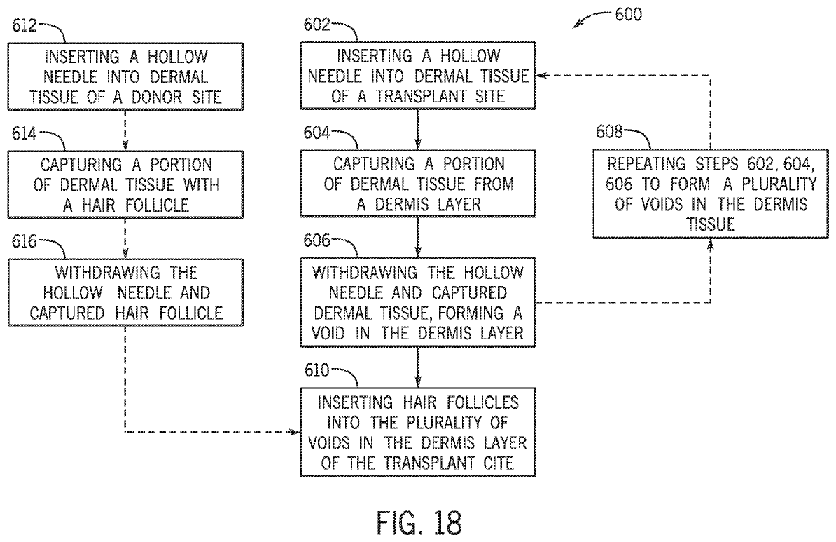

In this regard, for example, FIG. 18 illustrates an example method 600 for hair transplantation. At block 602, the method 600 can include inserting a hollow needle into dermal tissue of a transplant site configured to receive the hair transplantation. In some configurations, as also discussed above, the block 602 can include using a hollow needle with a cutting tip and a central lumen diameter between about 500 nm and about 0.5 mm.

At block 604, the method 600 can include capturing a portion of dermal tissue from a dermis layer within the central lumen. In some configurations, the hollow needle can include a protrusion within the central lumen near the cutting edge to facilitate the capturing of the dermal tissue. Additionally, or alternatively, in some configurations, the central lumen can be shaped to compress and retain the dermal tissue therein to facilitate the capturing thereof.

At block 606, the method 600 can further include withdrawing the hollow needle along with the portion of dermal tissue captured within the central lumen, forming a void in the dermis layer. Additionally, or optionally, at block 608, the method 600 can further include repeating the steps of blocks 602, 604, and 606 a plurality of times to create a plurality of voids in the dermis layer. In some configurations, the step of block 608, regarding repeating the steps of blocks 602, 604, and 606, can include performing the steps of blocks 602, 604, and 606 in parallel with a multi-needle apparatus including a plurality of hollow needles.

In some configurations, at block 610, the method 600 can further include inserting hair follicles into the plurality of voids in the dermis tissue of the transplant site without creating bunching of the dermis layer. Additionally, or optionally, as also discussed above, a hair follicle can be harvested from a donor site. For example, at block 612, the method 600 can include inserting a hollow needle into dermal tissue of a donor site, capturing a hair follicle therefrom (block 614), and withdrawing the hollow needle and with the hair follicle captured within the central lumen.

The previous description of the disclosed embodiments is provided to enable any person skilled in the art to make or use the invention. Various modifications to these embodiments will be readily apparent to those skilled in the art, and the generic principles defined herein may be applied to other embodiments without departing from the spirit or scope of the invention. Thus, the invention is not intended to be limited to the embodiments shown herein but is to be accorded the widest scope consistent with the principles and novel features disclosed herein.

1. A method for hair transplantation including steps comprising:
a) inserting a hollow needle into dermal tissue of a transplant site, the hollow needle having a cutting tip and a central lumen with a central lumen diameter between about 500 nm and about 0.5 mm;
b) capturing a portion of dermal tissue from a dermis layer within the central lumen;
c) withdrawing the hollow needle along with the portion of dermal tissue, forming a void in the dermis layer;
d) repeating steps a) through c) a plurality of times to create a plurality of voids in the dermis layer; and
e) inserting hair follicles into the plurality of voids in the dermis layer of the transplant site without creating bunching of the dermis layer.

2. The method of claim 1, wherein the hollow needle includes a protrusion within the central lumen configured to capture the portion of dermal tissue in the capturing step.

3. The method of claim 1, wherein step d includes performing steps a through c in parallel with a multi-needle apparatus including a plurality of hollow needles.

4. The method of claim 1, further comprising the steps of:
inserting the hollow needle into dermal tissue of a donor site;
capturing a portion of dermal tissue with a hair follicle within the central lumen; and
withdrawing the hollow needle along with the portion of dermal tissue.

5. The method of claim 1, wherein a grip is attached to the hollow needle and defines a maximum insertion distance between a lower grip surface and a most distal part of the cutting tip, the grip being configured to limit the distance the hollow needle can extend into the dermal tissue in the inserting step.

6. The method of claim 5, wherein the grip is configured to be moved relative to and along the hollow needle to modify the maximum insertion distance.

7. An apparatus for removing dermal tissue from a transplant site to receive hair transplants, comprising:
a hollow needle with a proximal end, a distal end, and a central lumen extending from the proximal end through the distal end, the distal end having an opening aligned with the central lumen to define a maximum cross-section dimension in the range of about 500 nm to about 0.5 mm and configured to cut into the dermal tissue to allow the dermal tissue to extend into the central lumen and to excise the dermal tissue from the transplant site to create a void sized to counteract bunching of the dermal tissue after receiving hair transplants; and
a grip coupled to the proximal end of the hollow needle and configured to aid in controlling the hollow needle while cutting and excising the dermal tissue.

8. The apparatus of claim 7, further comprising a cutting tip at the distal end that is obliquely angled relative to a longitudinal axis of the central lumen.

9. The apparatus of claim 8, wherein the angle of the cutting tip is about 45 degrees.

10. The apparatus of claim 7, wherein the grip defines a maximum insertion distance between a lower grip surface and a most distal part of the cutting tip, the grip being configured to limit the distance the hollow needle can extend into the dermal tissue.

11. The apparatus of claim 10, wherein the maximum insertion distance is the length of a portion of a hair follicle within the dermal tissue.

12. The apparatus of claim 10, wherein the maximum insertion distance is less than 5 mm.

13. The apparatus of claim 10, wherein the grip is movable relative to and along the hollow needle to alter the maximum insertion distance.

14. The apparatus of claim 7, further comprising a protrusion extending inward from the hollow needle into the central lumen, the protrusion configured to engage the dermal tissue entering the central lumen and retain the dermal tissue in the central lumen as the hollow needle is withdrawn from the transplant site to excise the dermal tissue from the transplant site.

15. The apparatus of claim 7, wherein the central lumen extends along a longitudinal axis of the hollow needle and the central lumen diameter decreases in dimension along the longitudinal axis from the cutting tip in a direction toward the proximal end.

16. The apparatus of claim 7, wherein the cross-section of the central lumen is diamond shaped.

17. The apparatus of claim 7, further comprising a plurality of hollow needles and wherein the grip forms a substrate having a plurality of holes in which the plurality of hollow needles are affixed.

18. The apparatus of claim 17, wherein the plurality of hollow needles are arranged in a rectangular pattern and spaced equidistantly in the substrate.

19. An apparatus for removing dermal tissue from a transplant site to receive hair transplants, comprising:
a hollow needle with a proximal end, a distal end, and a central lumen extending from the proximal end through the distal end, the distal end including a cutting tip with an opening diameter in the range of about 500 nm to about 0.5 mm and configured to cut into the dermal tissue to allow the dermal tissue to extend into the central lumen and to excise the dermal tissue from the transplant site;
a substrate in which the hollow needle is affixed;
a housing; and
a linkage system coupled to the housing;
wherein the substrate is configured to translate within the housing upon activation of the linkage system, which extends the hollow needle out of the housing.

20. The apparatus of claim 19, wherein the linkage system includes a slider-crank linkage that is activated by a switch on the housing, wherein when the switch rotates a crank at least half a full revolution per activation.

21. The apparatus of claim 19, further comprising a plurality of needles affixed to the substrate.
